(19) United States
(12) Patent Application Publication
(10) Pub. No.: US 2010/0330686 A1
(43) Pub. Date: Dec. 30, 2010

(54) NANOSENSOR FOR SUGAR DETECTION

(76) Inventor: Seung Bum PARK, Seoul (KR)

Correspondence Address: FOLEY & LARDNER LLP, 150 EAST GILMAN STREET, P.O. BOX 1497, MADISON, WI, 53701-1497, US

(21) Appl. No.: 12/493,631

(22) Filed: Jun. 29, 2009

(51) Int. Cl. G01N 33/50

(52) U.S. Cl. 436/94

(57) ABSTRACT

Nanosensors for carbohydrate detection are disclosed. The nanosensors include a nanoparticle conjugated to one or more boronic acid molecules or derivatives thereof and one or more pH sensitive materials. The nanosensors may be provided on a pH indicator paper in order to quickly assay samples, such as food samples.

### BACKGROUND

The present technology relates generally to the detection and quantification of carbohydrate analytes in samples, e.g., food samples.

Fruit producers and others often analyze the major constituents, such as sugar content and moisture, of certain agricultural products including sugar beets, grapes, and grapefruits. This analysis can be used for developing new hybrid species of crops. Moreover, with the advent of precision farming, it is desirable to obtain information relating to quality, such as sugar content, of an agricultural product being harvested along with yield (quantity) information.

### SUMMARY

In one aspect, the technology provides a carbohydrate sensing composition, i.e., a nanosensor, comprising: a nanoparticle; one or more boronic acid molecules or derivatives thereof associated with the nanoparticle; and one or more pH sensitive materials.

In some embodiments, the one or more boronic acid molecules or derivatives thereof are selected from the group consisting of: boronic acid; phenylboronic acid; p-nitrophenylboronic acid, 4-methoxyphenylboronic acid, α-naphthylboronic acid, 4-aminomethyl-2-N,N′-dimethylaminomet-hylphenylboronic acid, 3-fluoro-4-aminophenylboronic acid, 3,5-difluorophenylboronic acid, 2,4,6-trifluoropheylboronic acid, 3,5-dichlorophenylboronic acid, 2,4,6-trichloropheylboronic acid, 3-nitrophenylboronic acid, 4-N,N-dimethylpheylboronic acid, 3-methoxyphenylboronic acid, 2-methoxyphenylboronic acid, 3-fluorophenylboronic acid, 4-fluorophenylboronic acid, 2-fluorophenylboronic acid, 3-chlorophenylboronic acid, 4-chlorophenylboronic acid, 2-chlorophenylboronic acid, and their derivatives.

In some embodiments, the one more pH sensitive materials are selected from the group consisting of: Gentian violet (Methyl violet); Leucomalachite green; thymol blue; methyl yellow, bromophenol blue; congo red; methyl orange; bromocresol green; methyl red; azolitmin, bromocresol purple; bromothymol blue; phenol red; neutral red; naptholphtalein; thymolphthalein; alizarine yellow R; cresol red; m-cresol purple; xylenol orange; nitrazine yellow; rosolic acid; brilliant yellow; and chlorophenol red.

In some embodiments, the compositions further comprising a surface, wherein the one or more pH sensitive materials are immobilized on the surface. In one embodiment, the surface is a silica membrane or a paper disposed on a plastic substrate. In another embodiment, the plastic substrate comprises a thermoplastic film. In one embodiment, the pH sensitive material impregnates the surface.

In another aspect, the technology provides a carbohydrate sensing device comprising: a nanoparticle associated with one or more boronic acid molecules or derivatives thereof; one or more of pH-sensitive materials; and a surface being associated with the nanoparticle and the one or more of pH-sensitive materials.

In another aspect, the technology provides a carbohydrate sensing kit comprising the carbohydrate sensing device described above and instructions for using the device to detect carbohydrate in a sample.

In another aspect, the technology provides a method of assaying carbohydrate in a sample, comprising: contacting the sample with the carbohydrate sensing composition to form a reaction mixture; and observing a color change in the pH-sensitive material. In one embodiment, the one or more boronic acid molecules or derivatives thereof contain a combination of phenylboronic acid/boric acid. In one embodiment, the one or more pH sensitive materials contain a combination of Cresol red, Xylenol Orange, Nitrazine Yellow, Brilliant Yellow and Cresol Purple. In one embodiment, the observing step is conducted by quantitative or semi-quantitative colorimetry, densitometry, visible spectroscopy, or visual inspection. In some embodiments, the sample is a food sample.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram of an illustrative reaction of the nanoparticle-conjugated boronic acid with a carbohydrate.

FIG. 2 is a schematic diagram of an illustrative reaction of the nanoparticle-conjugated boronic acid with a carbohydrate to produce a color change on pH indicator paper.

### DETAILED DESCRIPTION

In the following detailed description, reference may be made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

Carbohydrate discrimination is highly challenging as a result of the similarity in functional groups among various types of carbohydrates. At present, technologies are available to determine the sugar content of sugar-containing agricultural products only under laboratory conditions or in a processing plant. However, it may be useful to obtain quality information during harvesting of fruits and vegetables in order to manage and plan crop production for consistent quality. Therefore, the present technology provides carbohydrate sensing devices and compositions to operate in a field environment and which are also capable of determining sugar content in a quick manner.

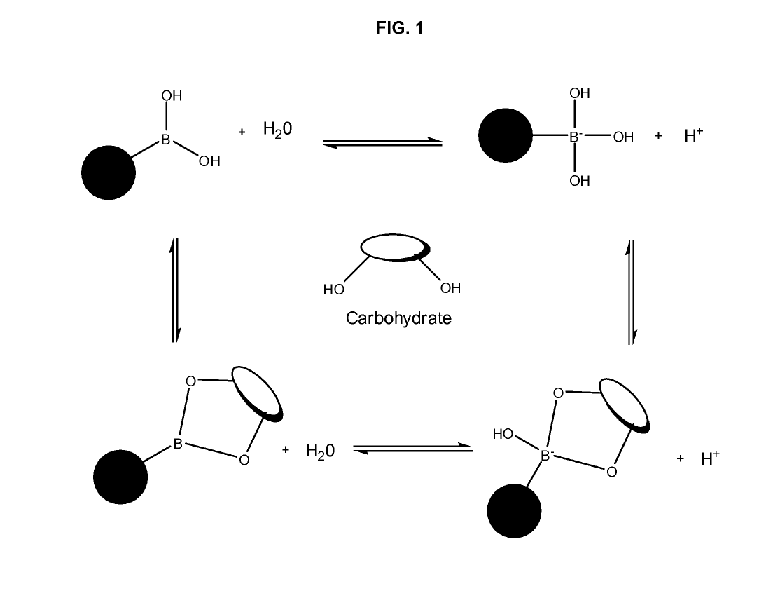

The present technology provides compositions, devices, methods and kits that can measure the carbohydrate content of various samples, including food samples, using a boric/boronic acid reagent coupled to a nanoparticle (See FIG. 1), which is in turn coupled to a substrate. Boronic acid forms reversible cyclic ester complexes with diols, which are abundant in carbohydrates, and undergoes an acid-base reaction with water as a conjugate acid (FIG. 1). Typically, boronic ester is more acidic than boronic acid, and ester formation is preferred at higher pH values by an equilibrium shift to the conjugate base, which releases a proton, thereby changing the pH of the composition.

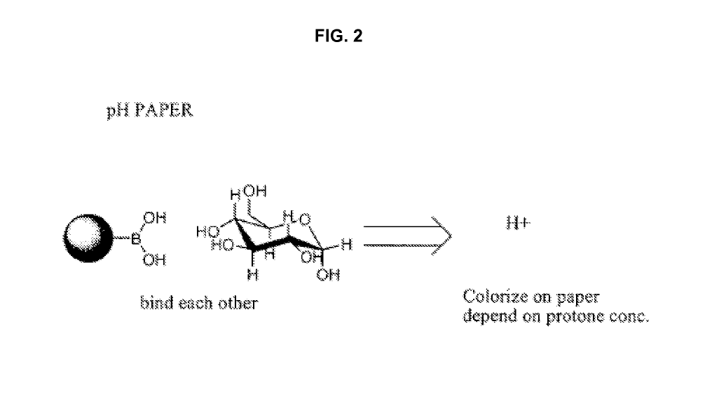

The nanoparticle and/or the substrate are further associated with pH sensitive materials, i.e., pH indicator or pH indicator dyes. The indicator dye can interact with the proton released upon formation of the boronic ester, resulting in a detectable color change. The type and quantity of the carbohydrates in the sample can then be observed by a color difference (See FIG. 2). A combination of the boric/boronic acid reagent coupled to a nanoparticle and multiple pH indicators can be used to discriminate between at least 23 different carbohydrates, including, but not limited to L-Sorbose; Palatinose; D-Fructose; D-Ribose; D-Xylose; D-Lyxose; Melibiose; D-Arabinose; D-Mannose; D-Galactose; D-Glucose; L-Rhamnose; L-Fucose; β-D-Lactose; D-Maltose; 2-Deoxy-D-ribose; myo-lnositol; 2-Deoxy-D-lyxohexose; D-Raffinose; D-Cellobiose; D-Trehelaose; D-Melezitose; and Sucrose. The use of the boric/boronic acid reagent has been described in Lee et al., “Colorimetric identification of carbohydrates by a pH indicator/pH change inducer ensemble,” Angew. Chem. Int. Ed. Engl., 45:6485-6487 (2006).

While not wishing to be limited by theory, the nanoparticle can provide a large surface area and extended stability for the reaction of the boric/boronic acid reagent with one or more carbohydrates in a sample. The nanoparticles provide enormous surface area compared to their weight, which provides enough space for the reaction with boronic acid reagents. One advantage of immobilization of boronic acid reagent on nanoparticle is enhanced sensitivity. The interaction between the boronic acid reagent and the carbohydrate is reversible; therefore, the detection is based on the equilibrium constant of the conjugated form of boronic acid and carbohydrate. In solution, the equilibrium between the free form and the conjugated form may not be high enough to permit detection. However, on the surface of nanoparticles, the carbohydrate can have an extended retention time with boronic acid because the effective molarity of boronic acid on the surface of the nanoparticles is increased. In fact, the effective concentration of the boronic acid moiety on the 2-dimensional surface of a nanoparticle is significantly larger than that in solution. Therefore, the equilibrium constant of the conjugated form of carbohydrate with boronic acid moiety on the surface of nanoparticle is much larger, which will enhance the sensitivity of boronic acid-based nanosensor. Nanoparticles can also provide a solid support for the boronic acid reagent to interact with its analytes (carbohydrates), but they are small enough to not to interfere with the reaction. Therefore, the reaction kinetics of boronic acid-linked nanoparticles are almost identical to that of solution reaction.

In some embodiments, the carbohydrate sensing device includes a pH change inducer, which is a boronic acid (or a derivative thereof) linked to the surface of a nanoparticle. In turn, the nanoparticle is linked to a pH sensitive material (e.g., a pH indicator paper) that is associated one or more pH indicator dyes. Alternatively, the nanoparticle may be functionalized directly with a pH indicator dye.

The present technology utilizes boric acid and/or boronic acid derivatives, which react with carbohydrates. The boronic acid is an aryl and alkyl substituted boric acid containing a carbon to boron chemical bond. The boronic acid includes but is not limited to phenylboronic acid; p-nitrophenylboronic acid, 4-methoxyphenylboronic acid, α-naphthylboronic acid, 4-aminomethyl-2-N,N′-dimethylaminomet-hylphenylboronic acid, 3-fluoro-4-aminophenylboronic acid, 3,5-difluorophenylboronic acid, 2,4,6-trifluoropheylboronic acid, 3,5-dichlorophenylboronic acid, 2,4,6-trichloropheylboronic acid, 3-nitrophenylboronic acid, 4-N,N-dimethylpheylboronic acid, 3-methoxyphenylboronic acid, 2-methoxyphenylboronic acid, 3-fluorophenylboronic acid, 4-fluorophenylboronic acid, 2-fluorophenylboronic acid, 3-chlorophenylboronic acid, 4-chlorophenylboronic acid, 2-chlorophenylboronic acid, and their derivatives. In one embodiment, multiple boronic acid derivatives may be used in combination of at least two or more. In another embodiment, the combination of boric acid/phenyboronic acid is used.

Different carbohydrates exhibit different binding constants to boronic acid. Moreover, the binding constants of different boronic acid derivatives to carbohydrates produce a unique pH change depending on the particular boronic acid derivative selected and the carbohydrate present in the sample. For example, in a sodium phosphate buffer (10 mM), boric acid and phenylboronic acid have pKₐ values of 9.2 and 8.8, respectively. In the absence of carbohydrate, the final pH of boric acid and phenylboronic acid are 8.0 and 7.9, respectively. Thus, the choice of a particular boronic acid derivative can provide a way to discriminate between a wide variety of carbohydrates using a variety of indicator dyes.

In one embodiment, the nanoparticles useful in the compositions and devices include, but are not limited to, polymeric nanoparticles, dendrimers, and metal nanoparticles, e.g., gold, silver, platinum, palladium, iron-gold alloy, iron-platinum alloy, iron oxide, cadmium selenide, cobalt ferrite, copper ferrite, nickel ferrite, zinc ferrite, and transition metal chalcogenides passivated by zinc sulfide. For example, metal nanoparticles may include gold or silver nanoparticles having a diameter that ranges from about 1 nm to about 4 nm. The diameter of the gold nanoparticles may vary. In some embodiments, the diameter of the gold nanoparticle is about 60 nm or less. This includes embodiments where the diameter is about 45 nm or less, or about 35 nm or less. In some embodiments, the nanoparticles are silica nanoparticle or silica-coated metallic nanoparticles. In some embodiments, the nanoparticles are (1) monodisperse organically passivated nanoparticles (“Synthesis and Characterization of monodisperse nanocrystals and close-packed nanocrystal assemblies” Annu. Rev. Mater. Sci. 2000. 30:545-610; “Ultra-large-scale syntheses of monodisperse nanocrystals” Nature Materials 2004, 3:891-895); (“A Simple Large-Scale Synthesis of Nearly Monodisperse Gold and Silver Nanoparticles with Adjustable Sizes and with Exchangeable Surfactants” Chem. Mater. 2004. 16:2509-2511); (3) monodisperse, starched silver nanoparticles (“Completely “Green” Synthesis and Stabilization of Metal Nanoparticles” J. Am. Chem. Soc. 2003, 125:13940-13941).

In some embodiments, the nanoparticles may include one or more functional groups, which are capable of reacting with one or more reactive groups on the boronic acid (or a derivative thereof) and/or pH indicator dye. The reactive groups may also be capable of reacting with linker molecules (described below), which provide a molecular link between the nanoparticle and the boronic acid and/or pH indicator. The reactive groups may naturally occur on the molecule, or the molecule may be modified to provide the reactive groups. A variety of reactive groups may be used, including, but not limited to thiol groups, carboxylic groups, and/or hydroxyl groups. By way of example, a molecule modified with one or more thiol groups can be coupled to a nanoparticle derivative with one or more thiol groups through the formation of a disulfide bond. Synthetic methods for modifying molecules to provide reactive groups and for reacting the boronic acid and/or pH indicator dye with the functionalized nanoparticles are well-known. For example, the functionalization for specific immobilization on gold nanoparticles is possible using a gold-thiol specific interaction or gold-carboxylic acid (bidentate or tridentate) specific interaction. Through the introduction of thiol or bi(tri-)carboxylic acid group on the moiety with appropriate linkers, it is possible to introduce various boronic acids and/or pH indicators using conventional synthetic organic methods.

Various conjugation methods can be applied, e.g., amide coupling, click chemistry, Staudinger ligation, Michael addition, hydrazone formation, disulfide bond formation, crosslinking of diamine with CDl (carboxydimide), and olefin metathesis using Grubbs 2^{nd} generation catalyst. For amide coupling, a carboxylic acid moiety and (primary or secondary) amine moiety can be linked to each other through an activated ester. For Click chemistry, an alkyne and azide moiety can be utilized in the presence of copper catalyst for the formation of triazole as a linker. For Staudinger ligation, an azide moiety can specifically link to an alkyl (or aryl)phosphine with high specificity. For hydrazone formation, aldehyde and hydrazine can form a hydrazone moiety with high efficiency. Disulfide bonds can be formed with two free sulfhydryl moieties on two reaction partners. CDl (carbonyl diimidazole) can serve as an efficient cross-linker of two amine moieties between two reaction partners. The olefin metathesis method can be utilized for the cross-linking of two alkene moieties in the presence of Grubbs 2^{nd} generation catalyst.

The present composition may further include one or more linker molecules bound to the functionalized nanoparticle at one end and bound to the boronic acid and/or pH indicator at the other end. The linker molecules provide a molecular link between the nanoparticle and/or the boronic acid and/or pH indicator molecules. In addition, the linker molecules may provide space between the nanoparticle and the boronic acid and/or pH indicator dye thereby maintaining and/or enhancing the chemical properties of the boronic acid and/or pH indicator dye. A variety of linker molecules may be used, including, but not limited to polyethylene glycol, piperazine, polyglycine, and polyproline. In the case of metallic nanoparticles, the desired linkers can be introduced on the surface of metal nanoparticles through bi-(tri-)dentate carboxylic acids on the molecules, which can efficiently immobilize the linker molecule on the surface. Silica nanoparticle and silica-coated metallic nanoparticle can be modified with aminopropyltriethoxysilane (APS) under ethanol condition, which can covalently introduce amino propyl moiety through oxy-silicon linkage, and the resulting amine can be chemically modified with various boronic acids and/or pH indicators.)

Like the molecules described above, the linker molecules may include reactive groups that are capable of reacting with the functional groups on the nanoparticles, the boronic acid, and/or the pH indicator dye. These reactive groups may naturally occur on the linker molecule, or the linker molecule may be modified to provide the reactive groups. Any of the reactive groups disclosed above may be used. Synthetic methods for modifying linker molecules to include such reactive groups and for coupling the linker molecules to the functionalized nanoparticles, the boronic acid, and/or the pH indicator dye are known. The linker molecules (e.g., polyethylene glycol, piperazine, polyglycine, and polyproline) can be incorporated into the above-mentioned linker chemistry. That is, these linker molecules can be placed in between the boronic acid reagents and/or pH indicator dyes and the functionalized nanoparticles.

In some embodiments, it is possible to introduce a linker on the surface of the nanoparticle and specifically modify the functional handle on the nanoparticle with various boronic acids and/or pH indicators. For instance, linkers with an amine moiety can be coupled with the activated carboxylic acid of the sugar sensor (boronic acid) or indicator using PyBOP, EDC, DCC, PyBrop, EDCl and other types of coupling agents. The linkers with an amine moiety can also be coupled with the amine moiety of the sugar sensor (boronic acid) or indicator using CDl (carbodiimide) and other types of crosslinking agents. The linkers with a thiol moiety can also be coupled with a maleimide moiety of the sugar sensor (boronic acid) or indicator. Other types of crosslinking agents may be used, e.g., iodoacetamide moiety or alkyl iodide moiety.

In an illustrative embodiment, the nanoparticle conjugated with boronic acid (or a derivative thereof) is immobilized on the surface of a pH indicator paper. For example, the pH indicator paper may include carboxylic acid groups on the surface, which can efficiently bind with metal nanoparticles. In the case of surface modified metal nanoparticles, a non-covalent interaction of the nanoparticles retains the nanoparticles on the surface for period of time sufficient to conduct the assay, i.e., at least about 5 seconds, at least about 10 seconds, at least about 30 seconds, at least about 1 minute, at least about 5 minutes, at least about 10 minutes, at least about 20 minutes or at least about 30 minutes. Alternatively, the nanoparticles can be conjugated to the surface using one or more of the conjugation chemistries described above.

In some embodiments, the compositions described herein include at least one pH sensitive material (e.g., an indicator dye). A pH indicator is a substance that changes color with a change in pH. Such indicators are usually weak acids or bases that ionize in solution to produce their conjugate bases or acids. A weak acid indicator (HIn) and its conjugate base (In⁻) exist in the following equilibrium:

HIn(aq)+H₂O [symbol] H₃O⁺+In⁻(aq)

The weak acid and conjugate base have different colors. At low pH, the concentration of H₃O⁺ is higher; the equilibrium above shifts to the acid (or HIn) side, and the color of the weak acid is seen. As the pH increases, the concentration of H₃O⁺ decreases, shifting the equilibrium to the conjugate base and changing the color of the solution accordingly. As the pH increases, the equilibrium above is shifted to the conjugate base side. Therefore, this type of indicator agent operates by sensing changes in paper pH corresponding to the amount and type of carbohydrate in the sample.

Table 1 shows a non-exhaustive list of several common pH indicators. Indicators usually exhibit intermediate colors at pH values inside the listed transition range. For example, phenol red exhibits an orange color between pH 6.8 and pH 8.4. The transition range may shift slightly depending on the concentration of the indicator in solution and on the temperature at which it is used.

TABLE 1
Illustrative pH Indicator Dyes

| | Low | Transition | High |
|---|---|---|---|
| Indicator | pH color | pH range | pH color |
| Gentian violet (Methyl violet) | yellow | 0.0-2.0 | blue-violet |
| Leucomalachite green (first | yellow | 0.0-2.0 | green |
| transition) | | | |
| Leucomalachite green (second | green | 11.6-14 | colorless |
| transition) | | | |
| Thymol blue (first transition) | red | 1.2-2.8 | yellow |
| Thymol blue (second transition) | yellow | 8.0-9.6 | blue |
| Methyl yellow | red | 2.9-4.0 | yellow |
| Bromophenol blue | yellow | 3.0-4.6 | purple |
| Congo red | Blue-violet | 3.0-5.0 | red |
| Methyl orange | red | 3.1-4.4 | orange |
| Bromocresol green | yellow | 3.8-5.4 | blue-green |
| Methyl red | red | 4.4-6.2 | yellow |
| Methyl red/Bromocresol green | red | 4.5-5.2 | green |
| Azolitmin | red | 4.5-8.3 | blue |
| Bromocresol purple | yellow | 5.2-6.8 | purple |
| Bromothymol blue | yellow | 6.0-7.6 | blue |
| Phenol red | yellow | 6.8-8.4 | purple |
| Neutral red | red | 6.8-8.0 | yellow |
| Naphtholphthalein | colorless to | 7.3-8.7 | greenish to |
| | reddish | | blue |
| Cresol Red | yellow | 7.2-8.8 | Reddish- |
| | | | purple |
| Phenolphthalein | colorless | 8.3-10.0 | Fuchsia |
| Thymolphthalein | colorless | 9.3-10.5 | Blue |
| Alizarine Yellow R | yellow | 10.2-12.0 | red |

Combinations of the indicator dyes may be used to provide additional detection capability. In one embodiment, the present composition contains as pH sensitive materials a combination of Cresol red, Xylenol Orange, Nitrazine Yellow, Brilliant Yellow and Cresol Purple. In another embodiment, the pH sensitive materials may or may not be associated with boric/boronic acid derivatives. In some embodiments, the pH sensitive materials may be associated with a substrate as described below. In one embodiment, the substrates are impregnated with the pH sensitive materials.

The compositions may further include a substrate to which a nanoparticle and/or pH indicator dye may be associated. The substrate includes, but is not limited to, pH indicator paper. In illustrative embodiments, the paper may be unsized paper, absorbent paper, fiber paper, or unsized absorbent fiber paper. In an embodiment, the paper (e.g., unsized absorbent fiber paper) has a weight of about 25 to about 70 g/m². In an embodiment, the paper includes fibers (e.g., long fibers) from coniferous trees.

The substrate may also be a plastic substrate, which can be any of a variety of plastics suitable for coupling to a nanosensor. Such plastics include known thermoplastics. For example, the thermoplastic substrate can include or be polyethylene, polypropylene, polyvinyl acetate, and/or ethylene vinyl acetate copolymer. In an embodiment, the plastic substrate includes or is a thermoplastic polymer film. In an embodiment, the plastic substrate is transparent or translucent. In an embodiment, the thermoplastic polymer film mounts the pH indicator paper onto adhesive paper.

As used herein, the term “thermoplastic” refers to a plastic that can once hardened be melted and reset. Suitable thermoplastics include, but are not limited to, polyamide, polyolefin (e.g., polyethylene, polypropylene, poly(ethylene-copropylene), poly(ethylene-coalphaolefin), polybutene, polyvinyl chloride, acrylate, acetate, and the like), polystyrenes (e.g., polystyrene homopolymers, polystyrene copolymers, polystyrene terpolymers, and styrene acrylonitrile (SAN) polymers), polysulfone, halogenated polymers (e.g., polyvinyl chloride, polyvinylidene chloride, polycarbonate, or the like, copolymers and mixtures of these materials, and the like Illustrative vinyl polymers include those produced by homopolymerization, copolymerization, terpolymerization, and like methods. Suitable homopolymers include polyolefins such as polyethylene, polypropylene, poly-1-butene, etc., polyvinylchloride, polyacrylate, substituted polyacrylate, polymethacrylate, polymethylmethacrylate, copolymers and mixtures of these materials, and the like. Suitable copolymers of alpha-olefins include ethylene-propylene copolymers, ethylene-hexylene copolymers, ethylene-methacrylate copolymers, ethylene-methacrylate copolymers, copolymers and mixtures of these materials, and the like. Thermoplastic polymers are generally not highly crosslinked, and have low melting and boiling points, low strength, and low ductility. In certain embodiments, suitable thermoplastics include polyethylene, polypropylene, polyvinyl acetate, ethylene vinyl acetate copolymer, copolymers and mixtures of these materials, and the like.

In one embodiment, the support is a solid non-absorbent material. In one embodiment, the support has a matte finish. Therefore, when the calorimetric change is read visually or by a reflective spectrophotometry, the support is highly reflective to increase color contrast. Such a support includes the above materials as well as finished metal foils. When the color change is read by transmission spectrophotometry, a transparent support may be used.

The support is typically coated with a reagent layer which contains the nanomaterial conjugated to a boronic acid (or derivative thereof) to ascertain the presence of the carbohydrate sought to be detected in the sample. The reagent layer may include a polymeric layer that contains a dialyzed latex polymer material. Such latex polymeric substances are well known in the art and include emulsions of polyvinyl compounds such as polyvinyl acetate, polyvinyl propionate, polyvinyl butyracetal, polyvinyl copolymers, and the like. The material may be a polyvinyl acetate-ethylene copolymer, although other latex emulsions can be used. The polymer is dialyzed by various techniques well known to the person of ordinary skill in the art.

In some embodiments, the nanodevice may be formed into test strips. Test strips can be made in any desired arrangement which optionally may include various additional layers.

Also provided herein are methods of assaying carbohydrates in a sample using the disclosed composition or devices. The assay includes quantitative and/or qualitative determination of the carbohydrates present in the sample but is not limited thereto. The methods involve contacting the sample with the disclosed composition to form a reaction mixture. The reaction mixture then is characterized by a certain color through pH sensitive materials as a result of a pH change in the reaction mixture. The pH change is induced by the interaction between the disclosed composition and carbohydrates and the degree of pH change varies depending on the types of carbohydrates present in the sample. The pH change is then indicated by a pH sensitive material as mentioned above, which develops a certain color in the pH range specific to each pH sensitive material as described in Table 1.

A certain color developed indicates the presence of carbohydrates in the sample, and its intensity indicates the amount or concentration present in the reaction mixture. No color indicates an absence of carbohydrates. Qualitative determination, such as presence and/or absence of a particular carbohydrate, or a determination of a particular carbohydrate present in a particular sample, or quantitative determination may also be done by comparing the detected color with standard color changes generated by reacting the disclosed composition with series of different known amount of a particular carbohydrate or a mixture thereof.

The methods also include observing a color in the reaction mixture. The color change may be determined/detected by any conventional spectrophotometer or color reference chart on a strip. In one embodiment, the absorption spectrum is measured before and after contacting the disclosed composition and a sample. The reaction mixture is characterized by a particular absorption spectrum which varies depending on the color each reaction mixture has developed, which in turn depends on the pH of the reaction mixture. In one embodiment, the absorbance is measured between about 350 to about 750 nm.

In some embodiments of the methods, the color change may be detected by direct visualization with the human eye using the color reference chart. The varying degrees of color on the color reference chart is calibrated to the color formed when different concentrations and/different types of the carbohydrates are reacted with the disclosed composition. The chart may take the form of a strip having a plurality of spots corresponding to each color. The user may visually compare and match the color developed with the corresponding color on the color reference chart. Thus, the user is informed of the presence of the carbohydrate and the approximate amount of carbohydrate present in a sample.

In one aspect, a nanosensor for detecting carbohydrates in samples is provided. The nanosensor includes one or more substrate, having at least one surface for establishing fluid communication with a sample to be monitored, and, immobilized within (e.g., by entrainment or chemical bonding) the substrate, one or more nanoparticle-bound boron/boronic acid (or derivative thereof) compound and one or more pH indicator compounds. The substrate may be, but is not limited to, a hydrophobic paper (e.g., silicone-treated filter paper), hydrophilic paper, hydrophilic paper with a hydrophobic coating, or a polymer matrix. The indicator compound(s) may be embedded within the paper or polymer matrix or covalently bonded to the backbone of the polymer.

In one aspect, a nanosensor for detecting carbohydrates in samples is provided. In one embodiment, nanoparticles conjugated to a boronic acid molecule (or derivative thereof) and a pH indicator dye are contacted with a carbohydrate-containing sample in solution. After mixing and allowing the reaction to proceed for a sufficient period of time, a color change is observed. The reaction may proceed, e.g., at least about 5 seconds, at least about 10 second, at least about 15 seconds, at least about 30 seconds, at least about 1 minute, at least about 2 minutes, at least about 5 minutes, at least about 10 minutes, at least about 20 minutes, or at least about 30 minutes.

In another embodiment, a substrate associated with a nanoparticle/boronic acid derivative and a pH indicator is contacted with a carbohydrate-containing sample. For example, the nanosensor may include one or more substrates having at least one surface for establishing fluid communication with a sample to be monitored, and, immobilized on and/or within (e.g., by entrainment or chemical bonding) the substrate, one or more nanoparticle-bound boronic acid (or derivative thereof) compound and one or more pH indicator compounds. The substrate may be, but is not limited to, test strips of a hydrophobic paper (e.g., silicone-treated filter paper), hydrophilic paper, hydrophilic paper with a hydrophobic coating, or a polymer matrix. The indicator compound(s) may be linked to the nanoparticles, entrained within the paper or polymer matrix, or covalently bonded to the backbone of the polymer.

The pH indicator dye can be deployed in various ways to create a sensing system useful to detect various carbohydrates. In one embodiment, the indicator is embedded within a hydrophobic, fibrous matrix such as silicone-treated filter paper, which may safely be brought into contact with food. Water-soluble compounds are not washed out of the matrix despite exposure to polar compounds; indeed, the treated paper shows indicator activity even following an aqueous wash. The compounds may be embedded, for example, by soaking the matrix in a solution of the indicator followed by drying. Other embodiments utilize a fibrous hydrophilic matrix, or a hydrophilic matrix having a hydrophobic coating.

In another approach, the indicator molecule is incorporated within a polymer matrix. This may be achieved by mixing the indicator with a prepolymer prior to reaction; polymerization entrains the indicator molecule within the polymer matrix, with sufficient surface exposure and/or polymer permeability to facilitate adequate interaction (leading to a visible color change) with carbohydrates in samples. Alternatively, the indicator may be covalently bonded to the polymer backbone itself using hydroxyl functional groups or acylation.

The amount of carbohydrate present in the fluid is determined by comparing the results of the assay performed on known standards. For example, when a test strip is read visually, it may be compared with a preprinted chart showing the color obtained when using carbohydrate solutions of known concentration. Similarly, when the reading is by use of a spectrophotometer, the concentration is obtained from a standard graph prepared by using standard sugar solutions of known concentrations.

In some embodiments, the carbohydrate-sensing device can be used to quantify the amount of carbohydrate in a sample. For example, the carbohydrates can be quantified by the absorption intensity and/or comparison of color changes associated with known standards. In illustrative embodiments, the carbohydrates in a food sample are characterized and/or quantified. The nanosensor may be used to test a variety of different types of food samples. Foods are generally classified into the following groups: milk and milk products; eggs and egg products; meat and meat products; fish, mollusks, and crustaceans; oils and fats; grains and grain products; pulses, seeds, kernels, nuts; vegetables and vegetable products; fruits and fruit products; chocolate products, confectionary; and spices and herbs. In an illustrative embodiment, the nanosensor is used to test vegetables and fruits, e.g., prior to or at the time of harvest, in order to assess their ripeness or quality. “Vegetables and vegetable products” include, but are not limited to, broccoli, cabbage, carrot, cauliflower, celery, corn, cucumber, eggplant, green pea, green pepper, iceberg lettuce, mushroom, onion, potato, spinach, squash, string bean, sweet potato, and tomato. “Fruits and fruit products” include, but are not limited to, apple, avocado, banana, blueberry, cantaloupe, grape, grapefruit, lemon, olive, orange, peach, pineapple, and strawberry.

The present disclosure further relates to a method of making a carbohydrate-sensing device. The method can include providing a substrate, coupling a nanoparticle comprising a boronic acid derivative and the substrate, and coupling a pH indicator dye to the substrate and/or the nanoparticle. The carbohydrate-sensing device can be any of those described herein.

Also within the scope of the disclosure are kits including the carbohydrate nanosensor and instructions for use. The kits are useful for detecting the presence of carbohydrates in a sample, e.g., a food sample (fruit, vegetables, etc.). For example, the kit can include: a pH-sensitive material conjugated to one or more pH change-inducers, wherein the one or more pH change-inducers include a nanoparticle linked to one or more boronic acid derivative molecules. The kit components, (e.g., reagents) can be packaged in a suitable container.

The kit can also contain a control sample or a series of control samples (i.e. carbohydrate “standards”), which can be assayed and compared to the test sample. Each component of the kit can be enclosed within an individual container and all of the various containers can be within a single package, along with instructions for interpreting the results of the assays performed using the kit. The kits of the invention may contain a written product on or in the kit container. The written product describes how to use the reagents contained in the kit. In several embodiments, the use of the reagents can be according to the methods described herein.

### EXAMPLES

Gold Nanoparticle Coupled with Boronic Acid and pH Indicator.

This is the proposed general approach for the immobilization of organic small molecules (e.g., boronic acids and/or pH indicators as carbohydrate sensors) on gold nanoparticles. Gold nanoparticles can be treated with linker molecules functionalized with one or more thiol groups. The specific self-assembled monolayer of a thiol moiety on the surface of a gold nanoparticle can be robustly immobilized the linker molecules through gold-thiol specific interaction. A variety of linker molecules may be used, including, but not limited to, e.g., polyethylene glycol, piperazine, polyglycine, and polyproline. Through the introduction of thiol moiety with appropriate linkers, various boronic acids and/or pH indicators can be introduced using conventional synthetic organic methods. In fact, the functional handle on the nanoparticle can be modified with various boronic acids and/or pH indicators. For instance, linkers with amine moiety can be coupled with activated carboxylic acid of sugar sensor (boronic acid) or pH sensor using PyBOP, EDC, DCC, PyBrop, EDCl and other types of coupling agents. The linkers with the amine moiety can also be coupled with amine moiety of sugar sensor (boronic acid) or pH sensor using CDl (carbodiimide) and other types of crosslinking agents. The linkers with thiol moiety can also be coupled with maleimide moiety of sugar sensor (boronic acid) or pH sensor or other types (iodoacetamide moiety or alkyl iodide moiety) of crosslinking agents.

### Preparation of Standards

One (1) mg of fructose or (other monosaccharides or disaccharides or starch) is dissolved in 1 ml of deionized water to give a final concentration of 1 mg/ml. Several dilutions of the solution are made with the final concentrations ranging from 1×10⁻⁴ to 0.1% w/v (or 1 to 1000 ppm). An aliquot (100 μl) of each dilution sample are mixed with small portion (0.01 to 10 mg) of the carbohydrate and are left at RT for 30 mins before the absorbance is measured by scanning the mixture between about 350 to about 750 nm. Alternatively, the reaction is conducted on a strip containing the disclosed composition in discrete spots and the strip is used as a color reference chart for visual detection.

### Analysis of Samples Containing Carbohydrates.

Juices from various produce, such as apples, pears, and water melons are extracted and aliquots of each sample are taken for analysis. The aliquot may be filtered or may be used unfiltered for simple visual detection. To identify the carbohydrates in the samples, each sample is incubated with a single sensor system (prepared as described above) or an array of detection systems on nanoparticles. Upon contact of samples with a one or more sensor system in an array format, an individual carbohydrate will show a change in color and absorbance in visible light. Therefore, the pattern recognition of color changes can provide the qualitative identification of carbohydrates that are present in the sample, through the comparison with color patterns of individual carbohydrate as a standard and the color reference chart as described above.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

### EQUIVALENTS

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 units refers to groups having 1, 2, or 3 units. Similarly, a group having 1-5 units refers to groups having 1, 2, 3, 4, or 5 units, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A carbohydrate sensing composition comprising:
a nanoparticle;
one or more boronic acid molecules or derivatives thereof associated with the nanoparticle; and
one or more pH sensitive materials.

2. The composition of claim 1, wherein the one or more boronic acid molecules or derivatives thereof are selected from the group consisting of: boronic acid;
phenylboronic acid; p-nitrophenylboronic acid, 4-methoxyphenylboronic acid, α-naphthylboronic acid, 4-aminomethyl-2-N,N′-dimethylaminomet-hylphenylboronic acid, 3-fluoro-4-aminophenylboronic acid, 3,5-difluorophenylboronic acid, 2,4,6-trifluoropheylboronic acid, 3,5-dichlorophenylboronic acid, 2,4,6-trichloropheylboronic acid, 3-nitrophenylboronic acid, 4-N,N-dimethylpheylboronic acid, 3-methoxyphenylboronic acid, 2-methoxyphenylboronic acid, 3-fluorophenylboronic acid, 4-fluorophenylboronic acid, 2-fluorophenylboronic acid, 3-chlorophenylboronic acid, 4-chlorophenylboronic acid, 2-chlorophenylboronic acid, and their derivatives.

3. The composition of claim 1, wherein the one more pH sensitive materials are selected from the group consisting of: Gentian violet (Methyl violet); Leucomalachite green; thymol blue; methyl yellow, bromophenol blue; congo red; methyl orange; bromocresol green; methyl red; azolitmin, bromocresol purple; bromothymol blue; phenol red; neutral red; naptholphtalein; thymolphthalein; alizarine yellow R; cresol red; m-cresol purple; xylenol orange; nitrazine yellow; rosolic acid; brilliant yellow; and chlorophenol red.

4. The composition of claim 1 further comprising a surface, wherein the one or more pH sensitive materials are immobilized on the surface.

5. The composition of claim 4, wherein the surface is a silica membrane or a paper disposed on a plastic substrate.

6. The composition of claim 5, wherein the plastic substrate comprises a thermoplastic film.

7. The composition of claim 4, wherein the pH sensitive material impregnates the surface.

8. A carbohydrate sensing device comprising:
a nanoparticle associated with one or more boronic acid molecules or derivatives thereof;
one or more of pH-sensitive materials; and
a surface being associated with the nanoparticle and the one or more of pH-sensitive materials.

9. The device of claim 8, wherein the one or more boronic acid molecules or derivatives thereof are selected from the group consisting of: boronic acid; phenylboronic acid; p-nitrophenylboronic acid, 4-methoxyphenylboronic acid, α-naphthylboronic acid, 4-aminomethyl-2-N,N′-dimethylaminomet-hylphenylboronic acid, 3-fluoro-4-aminophenylboronic acid, 3,5-difluorophenylboronic acid, 2,4,6-trifluoropheylboronic acid, 3,5-dichlorophenylboronic acid, 2,4,6-trichloropheylboronic acid, 3-nitrophenylboronic acid, 4-N,N-dimethylpheylboronic acid, 3-methoxyphenylboronic acid, 2-methoxyphenylboronic acid, 3-fluorophenylboronic acid, 4-fluorophenylboronic acid, 2-fluorophenylboronic acid, 3-chlorophenylboronic acid, 4-chlorophenylboronic acid, 2-chlorophenylboronic acid, and their derivatives.

10. The device of claim 8, wherein the one or more of pH-sensitive materials are selected from the group consisting of: Gentian violet (Methyl violet); Leucomalachite green; thymol blue; methyl yellow, bromophenol blue; congo red; methyl orange; bromocresol green; methyl red; azolitmin, bromocresol purple; bromothymol blue; phenol red; neutral red; naptholphtalein; thymolphthalein; alizarine yellow R; cresol red; m-cresol purple; xylenol orange; nitrazine yellow; rosolic acid; brilliant yellow; and chlorophenol red.

11. The device of claim 8, wherein the surface is a silica membrane or a paper disposed on a plastic substrate.

12. The device of claim 11, wherein the plastic substrate comprises a thermoplastic film.

13. The device of claim 11, wherein the pH sensitive material impregnates the surface.

14. A carbohydrate sensing kit comprising the carbohydrate sensing device of claim 8 and instructions for using the device to detect carbohydrate in a sample.

15. A method of assaying carbohydrate in a sample, comprising:
contacting the sample with the composition of claim 1 to form a reaction mixture; and
observing a color change in the pH-sensitive material.

16. The method of claim 15, wherein the one or more boronic acid molecules or derivatives thereof contain a combination of phenylboronic acid/boric acid.

17. The method of claim 15, wherein the one or more pH sensitive materials contain a combination of Cresol red, Xylenol Orange, Nitrazine Yellow, Brilliant Yellow and Cresol Purple.

18. The method of claim 15, wherein the observing step is conducted by quantitative or semi-quantitative colorimetry, densitometry, visible spectroscopy, or visual inspection.

19. The method of claim 15, wherein the sample is a food sample.
